(19) United States
(12) Patent Application Publication
(10) Pub. No.: US 2020/0342547 A1
(43) Pub. Date: Oct. 29, 2020

(54) SYSTEMS AND METHODS FOR FACILITATING ENGAGEMENT BETWEEN ENTITIES AND INDIVIDUALS

(71) Applicant: Harris S. Haith, Woodbridge, NJ (US)

(72) Inventor: Harris S. Haith, Woodbridge, NJ (US)

(21) Appl. No.: 16/849,180

(22) Filed: Apr. 15, 2020

Related U.S. Application Data
(60) Provisional application No. 62/837,304, filed on Apr. 23, 2019.

(51) Int. Cl. G06Q 50/00; G16H 40/67; G06Q 30/02; G06F 16/78; G06F 40/30; G06K 9/00; G06K 9/72; A61B 5/16

(52) U.S. Cl. CPC G06Q 50/01; G16H 40/67; G06Q 30/0215; A61B 5/167; G06F 40/30; G06K 9/00718; G06K 9/726; G06F 16/7867

(57) ABSTRACT

Disclosed herein is a method for facilitating engagement between entities and individuals. Accordingly, the method may include receiving at least one digital content associated with at least one entity form at least one individual device associated with at least one individual, analyzing the at least one digital content associated with the at least one entity, determining at least one fanaticism value associated with the at least one digital content based on the analyzing, determining at least one reward corresponding to the at least one individual based on the at least one fanaticism value, generating at least one reward notification corresponding to the at least one reward based on the determining of the at least one reward, transmitting the at least one reward notification to at least one presentation device.

The current application claims a priority to the U.S. Provisional Patent application Ser. No. 62/837,304 filed on Apr. 23, 2019.

### FIELD OF THE INVENTION

Generally, the present disclosure relates to the field of data processing. More specifically, the present disclosure relates to systems and methods for facilitating engagement between entities and individuals.

### BACKGROUND OF THE INVENTION

Sports and entertainment hold a significant impact on the lives of individuals from all around the world. Whether it be a sports team, athlete, or musician, fans everywhere show an unmatched passion and love for their favorite sports and entertainment icons.

Existing techniques for facilitating engagement between entities and individuals are deficient with regard to several aspects. For instance, current technologies do not enable a deeper engagement between fans and the sports/entertainment entities.

Therefore, there is a need for improved systems and methods for facilitating engagement between entities and individuals that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method for facilitating engagement between entities and individuals, in accordance with some embodiments. Accordingly, the method may include a step of receiving, using a communication device, at least one digital content associated with at least one entity form at least one individual device associated with at least one individual. Further, the method may include a step of analyzing, using a processing device, the at least one digital content associated with the at least one entity. Further, the method may include a step of determining, using the processing device, at least one fanaticism value associated with the at least one digital content based on the analyzing. Further, the method may include a step of determining, using the processing device, at least one reward corresponding to the at least one individual based on the at least one fanaticism value. Further, the method may include a step of generating, using the processing device, at least one reward notification corresponding to the at least one reward based on the determining of the at least one reward. Further, the method may include a step of transmitting, using the communication device, the at least one reward notification to at least one presentation device.

Further disclosed herein is a system for facilitating to engagement between entities and individuals, in accordance with some embodiments. Accordingly, the system may include a communication device, and a processing device. Further, the communication device may be configured for receiving at least one digital content associated with at least one entity form at least one individual device associated with at least one individual. Further, the communication device may be configured for transmitting at least one reward notification to at least one presentation device. Further, the processing device may be configured for analyzing the at least one digital content associated with the at least one entity. Further, the processing device may be configured for determining at least one fanaticism value associated with the at least one digital content based on the analyzing. Further, the processing device may be configured for determining at least one reward corresponding to the at least one individual based on the at least one fanaticism value. Further, the processing device may be configured for generating the at least one reward notification corresponding to the at least one reward based on the determining of the at least one reward.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating to engagement between entities and individuals, in accordance with some embodiments.

FIG. 3 is a flowchart of a method for facilitating engagement between entities and individuals, in accordance with some embodiments.

FIG. 4 is a flowchart of a method for facilitating the analysis of sensor data associated digital content, in accordance with some embodiments.

FIG. 5 is a flowchart of a method for facilitating the identification of content metadata associated the digital content, in accordance with some embodiments.

FIG. 6 is a flowchart of a method for facilitating the detection of at least one of a fanaticism polarity and a fanaticism intensity of textual content, in accordance with some embodiments.

FIG. 7 is a flowchart of a method for facilitating the detection of at least one of a fanaticism polarity and a fanaticism intensity of graphical content, in accordance with some embodiments.

FIG. 8 is a flowchart of a method for facilitating the detection of at least one of a fanaticism polarity and a fanaticism intensity of audio content, in accordance with some embodiments.

FIG. 9 is a flowchart of a method for facilitating the detection of at least one of a fanaticism polarity and a fanaticism intensity of video content, in accordance with some embodiments.

FIG. 10 is a flow diagram of a method for facilitating engagement between entities and individuals, in accordance with some embodiments.

FIG. 11 is a schematic showing entity list of a Global Fan Draft App, in accordance with some embodiments.

FIG. 12 is a block diagram of a computing device for implementing the methods disclosed herein, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems and methods for facilitating engagement between entities and individuals, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice-based interface, gesture-based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role-based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine-readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor, etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview: The present disclosure describes systems and methods for facilitating engagement between entities and individuals. Further, the present disclosure may serve to instruct the sports and entertainment industries to have a “deeper engagement” with its fans around the world through a mobile application. Further, the mobile application may include a Global Fan Draft (GFD) App that may allow the fans to post fanatical messages, photos, and videos about their favorite entertainer, musician or sports team.

With the introduction of growing technologies in internet and social media, the fans may be able to connect with their favorite sports and entertainment entities more than ever. Further, the present disclosure may serve to connect the fans with the sports and entertainment entities on a deeper level of engagement than ever before.

Each league, or genre, may be ranked by the number of total posts. Further, the fans with the best posts may be drafted by their favorite entertainment or sports entity as part of the “Global Fan Draft Fan Team”. Some other lucky fans drafted may be chosen to represent their team as part of the “Global Fan Draft All-Stars”. In addition, there may also be a “Global Fan Draft Fan of the Week and Month”, culminating with the “Global Fan Draft Most Fanatical Fan (MFF) Award”. Further, a user does not need to be drafted by a team in order to be selected for the GFD Fan of the Week, Month, or MFF Awards.

To join the “Global Fan Draft” frenzy, the user may: A. Log onto the “Global Fan Draft” website and create an account.

B. Download the GFB App.

C. Click on the “Global Fan Draft Logo” homepage.

D. Choose a favorite entertainer, musician, or sports team and click on that logo, and begin posting their photos, messages, and/or videos showing their love.

Further, the user may even have their name announced on TV, radio, or see their picture in the news or a Jumbotron during “Global Fan Draft Night”.

Users may enjoy the “Global Fan Draft App” experience even more by clicking on one of its fun features such as:

1. “Global Fan Draft—What Do You Know?” This feature may allow fans to answer a series of 5 historical questions in 2 minutes or less and win a promotional prize from a GFD sponsor (only one try per month per fan).

2. “Global Fan Draft All-Access”. This feature may allow fans to see the latest photo or video posted by their GFD rep with their favorite entertainer, musician or sports team.

Users may see photos and videos only available on the GFD All-Access link.

3. “Global Fan Draft Assist”. Get notifications about special offers and same day ticket discounts, and the latest industry news. Stream games, content, or music live, or see it later on-demand. Users may purchase tickets, merchandise, or see schedules.

4. “Global Fan Draft See more”. This feature may take users back to the “Global Fan Draft” website where there is a link to view the latest updates, info, and GFD posts in any sports leagues, musicians, or entertainment content.

5. “Global Fan Draft History”. This feature may allow users to search for the history of any sports league, entertainment content, or musician's history, past or current.

The Global Fan Draft Operation concept is as follows:

Page #1. Global Fan Draft NBA App Homepage

A. Image of GFD/NBA Version Logo

Page #2. Global Fan Draft—Create an Account Page

A. Name

B. Address

C. Telephone Number

D. Email Address

E. Favorite Team

F. Age

Page #3. NBA Team Logos Page

A. Images of all 30 NBA Team Logos

Page #4. Global Fan Draft Posting Page

A. Blank page with instructions to post a message, photo. Or video of the user's favorite team. (With “Next Page” located at the bottom)

Page #5. Global Fan Draft App Features Link Page

A. GFD “All Access”. Upon selection a page may open to a title caption describing whatever photo and/or videos that appear on the page at that time. Read comments under photos or (to view) click on the videos exclusive to GFD.

B. Global Fan Draft “How Much Do You Know About Your Team”. Upon selection, a page opens to display a list of 5 questions concerning that user's favorite team's history that must be answered in 60 seconds or less. The page closes automatically after 1 minute and locks that user out for 30 days if they fail to answer the questions correctly. All fans who win receive promotional prizes.

C. Global Fan Draft “Pro Sports History”. Upon selection, a page may open to display every professional sport in captions.

GFD/NBA All Star Game Tournament Proposal:

The GFD/NBA All Star Game Tournament would replace the existing All-Star weekend format, with a tournament consisting of 4 teams: 2 from the Eastern Conference and 2 from the Western Conference.

The Eastern Conference teams would be named the Atlantic (Division) All Stars, and the Southeast (Division) All Stars. The Western Conference teams would be named the Pacific (Division) All Stars, and the West (Division) All Stars. (Note: The West Division All Stars would be a combination of players from the Southwest and the Northwest Division All Star players).

Each player of the winning team would receive one-million dollars, provided by the tournament sponsors.

Tournament format:

A. Friday: Atlantic All Stars versus West All Stars

B. Saturday: Pacific All Stars versus Southeast All Stars

C. Sunday: Championship Game (Friday winner versus Saturday winner

Special events include every team would be represented by its GFD Fan Team during the weekend and game, in addition to the GFD All Star Fan Teams. (Note: Fan teams may compete and represent their teams in e-sports and/or fantasy tournaments, and the best costume and exhibit competitions. Each winner would receive a 550,000 cash prize from sponsors.

Global Fan Draft Mobile entity list may include, but is not limited to movies, television, superheroes, NFL, NBA, MLB, soccer, NCAAM, MMA, NHL, MCAAW, NCAAF, tennis, golf, WNBA, NBA G League, boxing, special Olympics, X-games, WWE, e-sports, chalk, analytics, NCAA softball, NASCAR, F1, racing, Olympics, horse, RN FB, NCAA, LLWS, CFL, rugby, cricket, BMX, lacrosse, track and field, rodeo, motor cross, music artists, world cup, drag racing, boat racing, dog shows and monster truck.

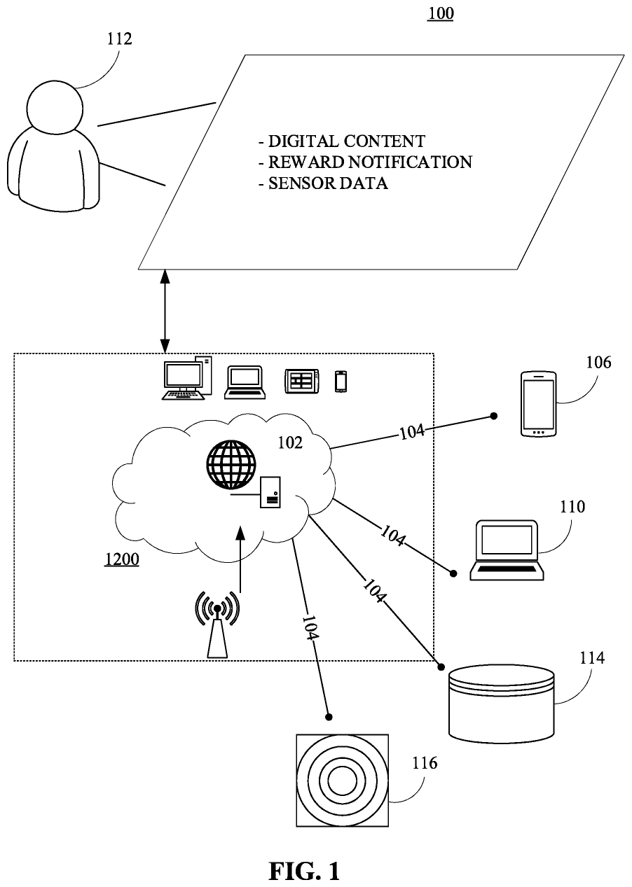

FIG. 1 is an illustration of an online platform 100 consistent with various embodiments of the present disclosure. By way of non-limiting example, the online platform 100 to facilitate engagement between entities and individuals may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 1200.

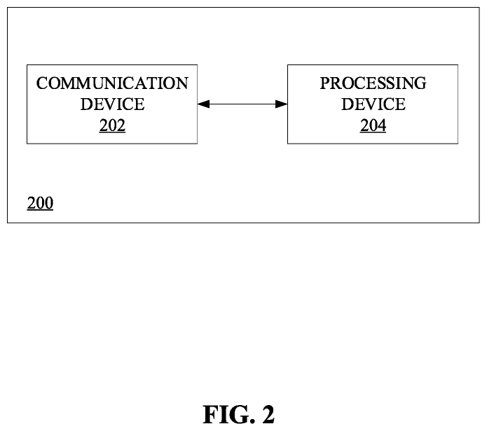

FIG. 2 is a block diagram of a system 200 for facilitating to engagement between entities and individuals, in accordance with some embodiments. Accordingly, the system 200 may include a communication device 202, and a processing device 204.

Further, the communication device 202 may be configured for receiving at least one digital content associated with at least one entity form at least one individual device associated with at least one individual. Further, the at least one digital content may include textual content, graphical content, audio content, video content, etc. Further, the at least one entity may be include a sports entity, an entertainment entity, etc. Further, the sports entity may include an entity related to sports and competitions such as, but not limited to, basketball, cricket, soccer, rugby, baseball, hockey, football, racing, Olympics, horse racing, boat racing, NBA G League, boxing, special Olympics, etc. Further, the entertainment entity may include entities related to, but not limited to, movies, television, superheroes, WWE, dog shows, etc. Further, the at least one individual device may include a mobile device 106 (such as a smartphone, a laptop, a tablet computer etc.), other electronic devices 110 (such as desktop computers, server computers etc.). Further, the communication device 202 may be configured for transmitting at least one reward notification to at least one presentation device. Further, the at least one presentation device may include a TV, a radio, a Jumbotron, a billboard, etc.

Further, the processing device 204 may be configured for analyzing the at least one digital content associated with the at least one entity. Further, the processing device 204 may be configured for determining at least one fanaticism value associated with the at least one digital content based on the analyzing. Further, the at least one fanaticism value may represent an enthusiasm level of the at least one individual. Further, the processing device 204 may be configured for determining at least one reward corresponding to the at least one individual based on the at least one fanaticism value. Further, the at least one reward may include a cash prize, a promotional prize, etc. Further, the at least one reward may be provided by tournament/competition sponsors. Further, the promotional prize may include displaying an individual's name on a website. Further, the processing device 204 may be configured for generating the at least one reward notification corresponding to the at least one reward based on the determining of the at least one reward.

Further, in some embodiments, the communication device 202 may be further configured for receiving at least one sensor data associated with the at least one digital content from at least one sensor. Further, the at least one sensor may be configured for generating the at least one sensor data based on at least one of a physical state and a physiological state of the at least one individual. Further, the processing device 204 may be further configured for analyzing the at least one sensor data associated. Further, the determining of the at least one fanaticism value may be based on the analyzing of the at least one sensor data. Further, in some embodiments, the at least one sensor may be comprised in the at least one individual device. Further, in some embodiments, the at least one sensor may be comprised in at least one wearable device. Further, the at least one individual wears the at least one wearable device. Further, the at least one wearable device may include a smartwatch, a smart lens, etc.

Further, in some embodiments, the processing device 204 may be further configured for comparing the at least one fanaticism value and at least one predetermined fanaticism value. Further, the determining of the at least one reward corresponding to the at least one individual may be based on the comparing.

Further, in some embodiments, the processing device 204 may be further configured for identifying at least one content metadata associated with the at least one digital content.

Further, the processing device 204 may be further configured for analyzing the at least one content metadata. Further, the determining of the at least one fanaticism value may be based on the analyzing of the at least one content metadata.

Further, in some embodiments, the at least one digital content may include at least one textual content. Further, the analyzing may include processing the at least one textual content through textual analysis. Further, the processing device 204 may be further configured for interpreting the at least one textual content through semantic analysis based on the processing. Further, the processing device 204 may be further configured for detecting at least one of a fanaticism polarity and a fanaticism intensity of the at least one textual content through sentiment analysis based on the interpreting. Further, the fanaticism polarity may include positive and negative. Further, the fanaticism intensity may provide a measure of intensity/height of fanaticism. Further, the determining of the at least one fanaticism value may be based on the detecting.

Further, in some embodiments, the at least one digital content may include at least one graphical content. Further, the analyzing may include processing the at least one graphical content through image analysis. Further, the processing device 204 may be further configured for interpreting the at least one graphical content through image-semantic analysis based on the processing. Further, the processing device 204 may be further configured for detecting at least one of a fanaticism polarity and a fanaticism intensity of the at least one graphical content through image-sentiment analysis based on the interpreting. Further, the fanaticism polarity may include positive and negative. Further, the fanaticism intensity may provide a measure of intensity/height of fanaticism. Further, the determining the at least one fanaticism value may be based on the detecting.

Further, in some embodiments, the at least one digital content may include at least one audio content. Further, the analyzing may include processing the at least one audio content through audio analysis. Further, the processing device 204 may be further configured for interpreting the at least one audio content through audio-semantic analysis based on the processing. Further, the processing device 204 may be further configured for detecting at least one of a fanaticism polarity and a fanaticism intensity of the at least one audio content through audio-sentiment analysis based on the interpreting. Further, the fanaticism polarity may include positive and negative. Further, the fanaticism intensity may provide a measure of intensity/height of fanaticism. Further, the determining the at least one fanaticism value may be based on the detecting.

Further, in some embodiments, the at least one digital content may include at least one video content. Further, the analyzing may include processing the at least one video content through video analysis. Further, the processing device 204 may be further configured for interpreting the at least one video content through video-semantic analysis based on the processing. Further, the processing device 204 may be further configured for detecting at least one of a fanaticism polarity and a fanaticism intensity of the at least one video content through video-sentiment analysis based on the interpreting. Further, the fanaticism polarity may include positive and negative. Further, the fanaticism intensity may provide a measure of intensity/height of fanaticism. Further, the determining the at least one fanaticism value may be based on the detecting.

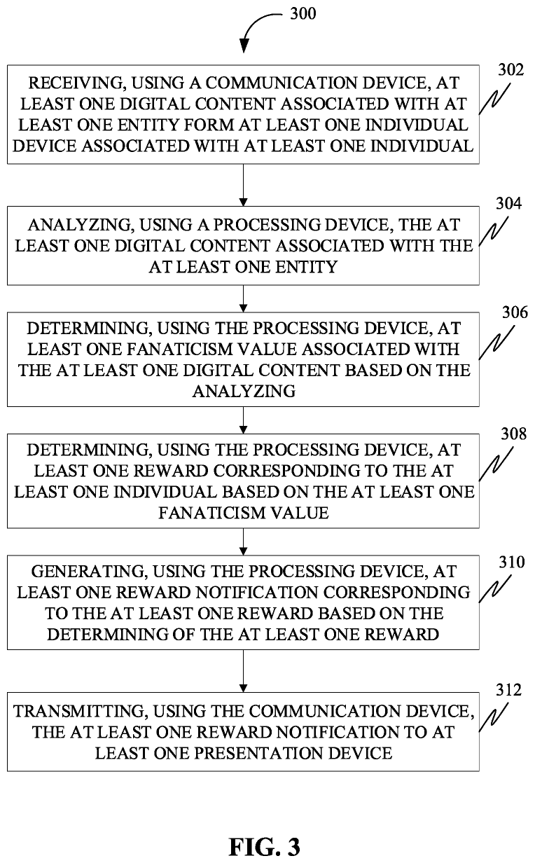

FIG. 3 is a flowchart of a method 300 for facilitating engagement between entities and individuals, in accordance with some embodiments. Accordingly, at 302, the method 300 may include a step of receiving, using a communication device, at least one digital content associated with at least one entity form at least one individual device associated with at least one individual. Further, the at least one digital content may include textual content, graphical content, audio content, video content, etc. Further, the at least one entity may be associated with a sports entity, an entertainment entity, etc. Further, the sports entity may include an entity related to sports/competitions such as, but not limited to, basketball, cricket, soccer, rugby, baseball, hockey, football, racing, Olympics, horse racing, boat racing, NBA G League, boxing, special Olympics, etc. Further, the entertainment entity may include an entity related to, but not limited to, movies, television, superheroes, WWE, dog shows, etc. Further, the at least one individual device may include a mobile device 106 (such as a smartphone, a laptop, a tablet computer etc.), other electronic devices 110 (such as desktop computers, server computers etc.).

Further, at 304, the method 300 may include a step of analyzing, using a processing device, the at least one digital content associated with the at least one entity.

Further, at 306, the method 300 may include a step of determining, using the processing device, at least one fanaticism value associated with the at least one digital content based on the analyzing. Further, the at least one fanaticism value may provide a measure of an enthusiasm level of the at least one individual.

Further, at 308, the method 300 may include a step of determining, using the processing device, at least one reward corresponding to the at least one individual based on the at least one fanaticism value. Further, the at least one reward may include a cash prize, a promotional prize, etc. Further, the at least one reward may be provided by tournament sponsors. Further, the promotional prize may include displaying an individual's name on a website.

Further, at 310, the method 300 may include a step of generating, using the processing device, at least one reward notification corresponding to the at least one reward based on the determining of the at least one reward.

Further, at 312, the method 300 may include a step of transmitting, using the communication device, the at least one reward notification to at least one presentation device.

Further, the at least one presentation device may include a TV, a radio, a Jumbotron, a billboard, etc.

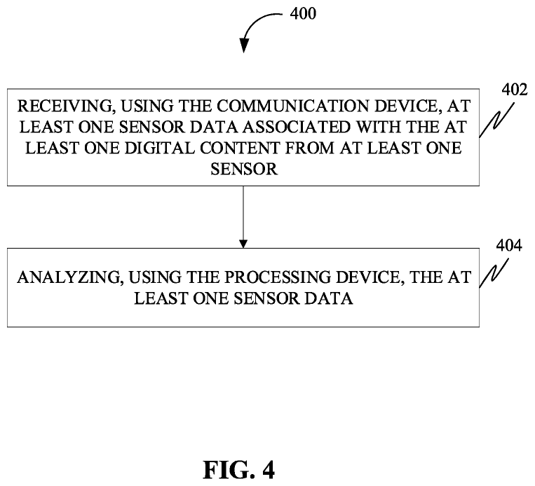

FIG. 4 is a flowchart of a method 400 for facilitating the analysis of sensor data associated digital content, in accordance with some embodiments. Accordingly, at 402, the method 400 may include a step of receiving, using the communication device, at least one sensor data associated with the at least one digital content from at least one sensor. Further, the at least one sensor may be configured for generating the at least one sensor data based on at least one of a physical state and a physiological state of the at least one individual.

Further, at 404, the method 400 may include a step of analyzing, using the processing device, the at least one sensor data. Further, the determining of the at least one fanaticism value may be based on the analyzing of the at least one sensor data.

Further, in some embodiments, the at least one sensor may be comprised in the at least one individual device. Further, in some embodiments, the at least one sensor may be comprised in at least one wearable device. Further, the at least one individual wears the at least one wearable device. Further, the at least one wearable device may include a smartwatch, a smart lens, etc.

Further, in some embodiments, the method 400 further may include comparing, using the processing device, the at least one fanaticism value and at least one predetermined fanaticism value. Further, the determining of the at least one reward corresponding to the at least one individual may be based on the comparing.

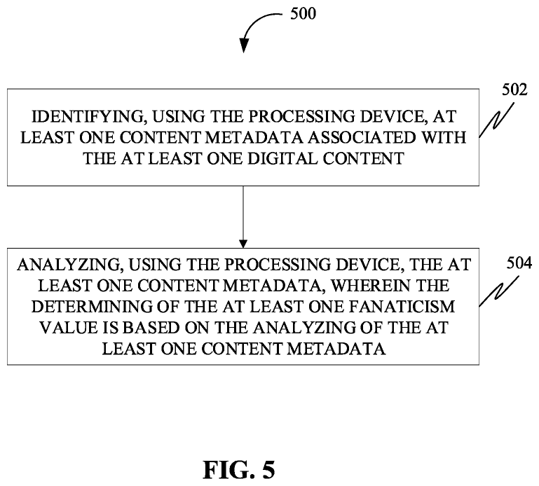

FIG. 5 is a flowchart of a method 500 for facilitating the identification of content metadata associated the digital content, in accordance with some embodiments. Accordingly, at 502, the method 500 may include a step of identifying, using the processing device, at least one content metadata associated with the at least one digital content.

Further, at 504, the method 500 may include a step of analyzing, using the processing device, the at least one content metadata. Further, the determining of the at least one fanaticism value may be based on the analyzing of the at least one content metadata.

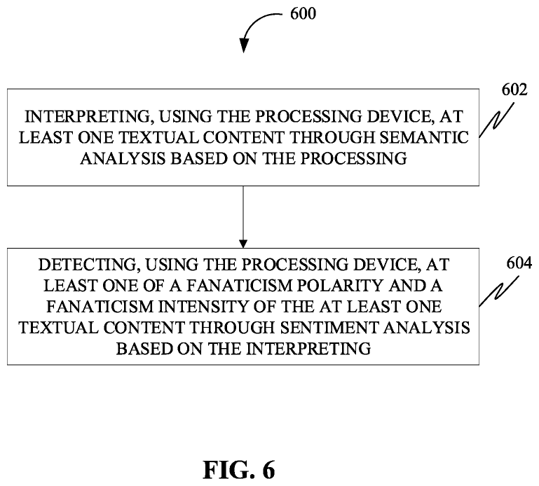

FIG. 6 is a flowchart of a method 600 for facilitating the detection of at least one of a fanaticism polarity and a fanaticism intensity of textual content, in accordance with some embodiments. Further, in some embodiments, the at least one digital content may include at least one textual content. Further, the analyzing may include processing the at least one textual content through textual analysis.

Accordingly, at 602, the method 600 may include a step of interpreting, using the processing device, the at least one textual content through semantic analysis based on the processing.

Further, at 604, the method 600 may include a step of detecting, using the processing device, at least one of a fanaticism polarity and a fanaticism intensity of the at least one textual content through sentiment analysis based on the interpreting. Further, the fanaticism polarity may include positive and negative. Further, the fanaticism intensity may provide a measure of intensity/height of fanaticism. Further, the determining of the at least one fanaticism value may be based on the detecting.

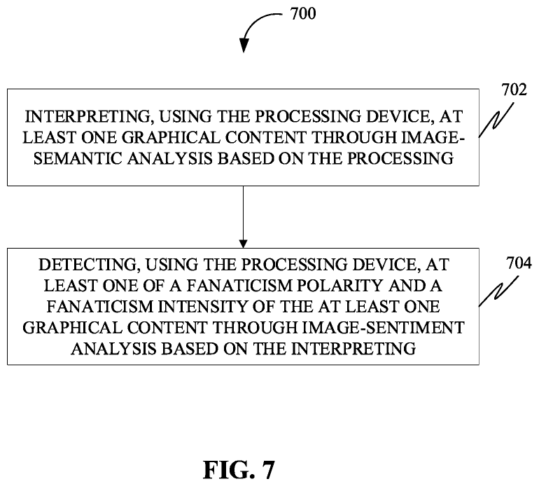

FIG. 7 is a flowchart of a method 700 for facilitating the detection of at least one of a fanaticism polarity and a fanaticism intensity of graphical content, in accordance with some embodiments. Further, in some embodiments, the at least one digital content may include at least one graphical content. Further, the analyzing may include processing the at least one graphical content through image analysis.

Accordingly, at 702, the method 700 may include a step of interpreting, using the processing device, the at least one graphical content through image-semantic analysis based on the processing.

Further, at 704, the method 700 may include a step of detecting, using the processing device, at least one of a fanaticism polarity and a fanaticism intensity of the at least one graphical content through image-sentiment analysis based on the interpreting. Further, the fanaticism polarity may include positive and negative. Further, the fanaticism intensity may provide a measure of intensity/height of fanaticism. Further, the determining the at least one fanaticism value may be based on the detecting.

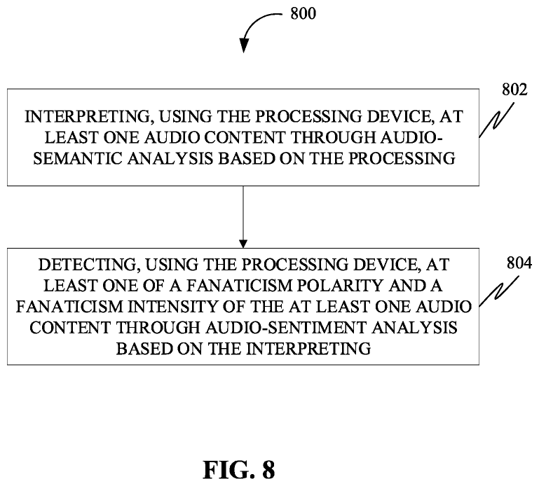

FIG. 8 is a flowchart of a method 800 for facilitating the detection of at least one of a fanaticism polarity and a fanaticism intensity of audio content, in accordance with some embodiments. Further, in some embodiments, the at least one digital content may include at least one audio content. Further, the analyzing may include processing the at least one audio content through audio analysis.

Accordingly, at 802, the method 800 may include a step of interpreting, using the processing device, the at least one audio content through audio-semantic analysis based on the processing.

Further, at 804, the method 800 may include a step of detecting, using the processing device, at least one of a fanaticism polarity and a fanaticism intensity of the at least one audio content through audio-sentiment analysis based on the interpreting Further, the fanaticism polarity may include positive and negative. Further, the fanaticism intensity may provide a measure of intensity/height of fanaticism. Further, the determining the at least one fanaticism value may be based on the detecting.

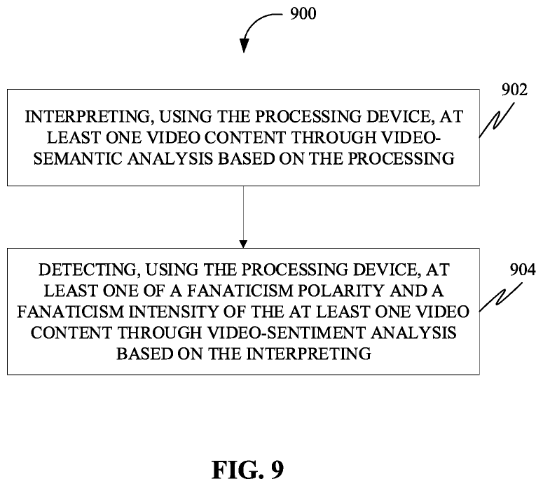

FIG. 9 is a flowchart of a method 900 for facilitating the detection of at least one of a fanaticism polarity and a fanaticism intensity of video content, in accordance with some embodiments. Further, in some embodiments, the at least one digital content may include at least one video content. Further, the analyzing may include processing the at least one video content through video analysis.

Accordingly, at 902, the method 900 may include a step of interpreting, using the processing device, the at least one video content through video-semantic analysis based on the processing.

Further, at 904, the method 900 may include a step of detecting, using the processing device, at least one of a fanaticism polarity and a fanaticism intensity of the at least one video content through video-sentiment analysis based on the interpreting. Further, the fanaticism polarity may include positive and negative. Further, the fanaticism intensity may provide a measure of intensity/height of fanaticism. Further, the determining the at least one fanaticism value may be based on the detecting.

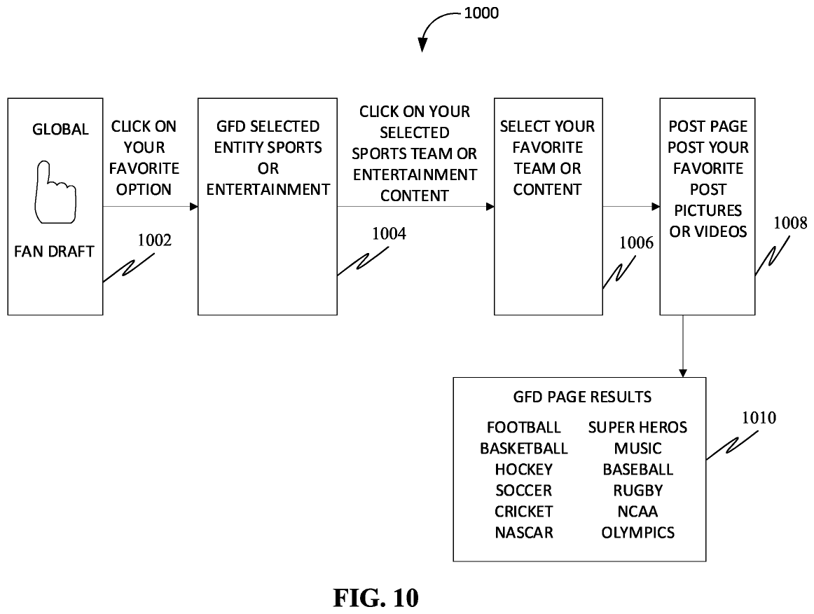

FIG. 10 is a flow diagram of a method 1000 for facilitating engagement between entities and individuals, in accordance with some embodiments. Further, at 1002, the method 1000 may include a step of downloading a Global Fan Draft (GFD) mobile application to access home page and clicking on a selection. Further, at 1004, the method 1000 may include a step of clicking on a favorite option. Further, the favorite option may include sports or entertainment. Further, at 1006, the method 1000 may include a step of selecting favorite sports team or entertainment content. Further, at 1008, the method 1000 may include a step of posting favorite pictures and videos. Further, at 1010, the method 1000 may include a step of displaying results on a company website. Further, the company website may be “www.globalfandraft.com”. Further, the company website may include a GFD result page. Further, the GFD result page may display results for football, basketball, hockey, soccer, cricket, Nascar, superheroes, music, baseball, rugby, NCAA, Olympics, etc.

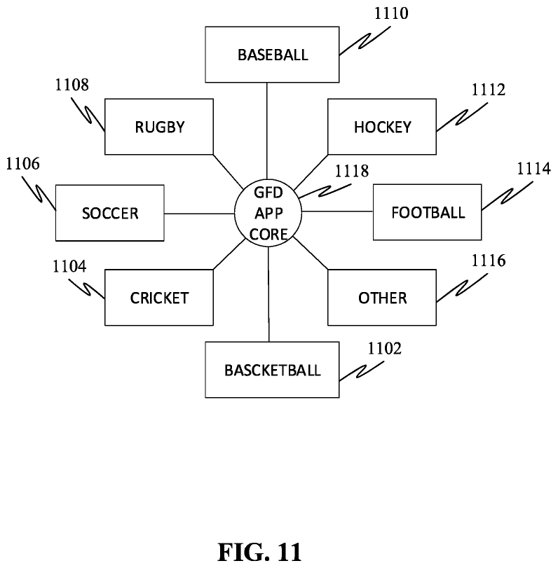

FIG. 11 is a schematic showing entity list of a Global Fan Draft App, in accordance with some embodiments. Further, a Global Fan Draft App core 1118 may include basketball 1102, cricket 1104, soccer 1106, rugby 1108, baseball 1110, hockey 1112, football 1114, and other 1116. Further, a method of signing in into the Global Fan Draft App core may include a step of creating an account. Further, the method may include a step of selecting desired app links from the Global Fan Draft App core. Further, the method may include a step of entering posts, videos, or photographs. Further, the method may be a part of the Global Fan Draft, all-star selection, and fan MVP selection process.

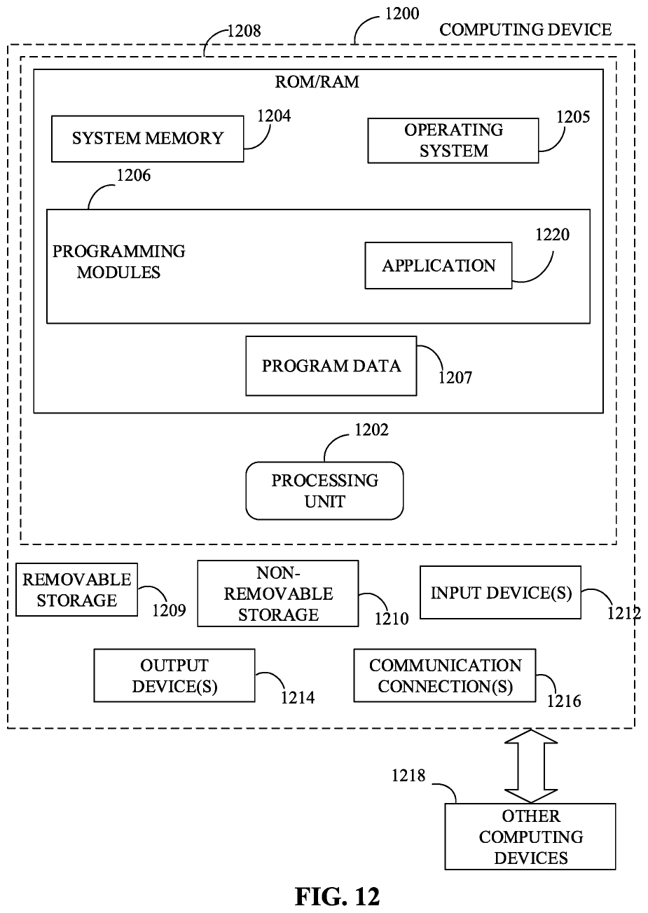

With reference to FIG. 12, a system consistent with an embodiment of the disclosure may include a computing device or cloud service, such as computing device 1200. In a basic configuration, computing device 1200 may include at least one processing unit 1202 and a system memory 1204. Depending on the configuration and type of computing device, system memory 1204 may comprise, but is not limited to, volatile (e.g. random-access memory (RAM)), non-volatile (e.g. read-only memory (ROM)), flash memory, or any combination. System memory 1204 may include operating system 1205, one or more programming modules 1206, and may include a program data 1207. Operating system 1205, for example, may be suitable for controlling computing device 1200's operation. In one embodiment, programming modules 1206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 12 by those components within a dashed line 1208.

Computing device 1200 may have additional features or functionality. For example, computing device 1200 may also include additional data storage devices (removable and/or non-removable) such as, for example, magnetic disks, optical disks, or tape. Such additional storage is illustrated in FIG. 12 by a removable storage 1209 and a non-removable storage 1210. Computer storage media may include volatile and non-volatile, removable and non-removable media implemented in any method or technology for storage of information, such as computer-readable instructions, data structures, program modules, or other data. System memory 1204, removable storage 1209, and non-removable storage 1210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1200. Any such computer storage media may be part of device 1200. Computing device 1200 may also have input device(s) 1212 such as a keyboard, a mouse, a pen, a sound input device, a touch input device, a location sensor, a camera, a biometric sensor, etc. Output device(s) 1214 such as a display, speakers, a printer, etc. may also be included. The aforementioned devices are examples and others may be used. Computing device 1200 may also contain a communication connection 1216 that may allow device 1200 to communicate with other computing devices 1218, such as over a network in a distributed computing environment, for example, an intranet or the Internet. Communication connection 1216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1204, including operating system 1205. While executing on processing unit 1202, programming modules 1206 (e.g., application 1220 such as a media player) may perform processes including, for example, one or more stages of methods, algorithms, systems, applications, servers, databases as described above. The aforementioned process is an example, and processing unit 1202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following:

an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A method for facilitating engagement between entities and individuals, the method comprising:
receiving, using a communication device, at least one digital content associated with at least one entity form at least one individual device associated with at least one individual;
analyzing, using a processing device, the at least one digital content associated with the at least one entity;
determining, using the processing device, at least one fanaticism value associated with the at least one digital content based on the analyzing;
determining, using the processing device, at least one reward corresponding to the at least one individual based on the at least one fanaticism value;
generating, using the processing device, at least one reward notification corresponding to the at least one reward based on the determining of the at least one reward; and
transmitting, using the communication device, the at least one reward notification to at least one presentation device.

2. The method of claim 1 further comprising:
receiving, using the communication device, at least one sensor data associated with the at least one digital content from at least one sensor, wherein the at least one sensor is configured for generating the at least one sensor data based on at least one of a physical state and a physiological state of the at least one individual; and
analyzing, using the processing device, the at least one sensor data, wherein the determining of the at least one fanaticism value is based on the analyzing of the at least one sensor data.

3. The method of claim 2, wherein the at least one sensor is comprised in the at least one individual device.

4. The method of claim 2, wherein the at least one sensor is comprised in at least one wearable device, wherein the at least one individual wears the at least one wearable device.

5. The method of claim 1 further comprising comparing, using the processing device, the at least one fanaticism value and at least one predetermined fanaticism value, wherein the determining of the at least one reward corresponding to the at least one individual is based on the comparing.

6. The method of claim 1 further comprising:
identifying, using the processing device, at least one content metadata associated with the at least one digital content; and
analyzing, using the processing device, the at least one content metadata, wherein the determining of the at least one fanaticism value is based on the analyzing of the at least one content metadata.

7. The method of claim 1, wherein the at least one digital content comprises at least one textual content, wherein the analyzing comprises processing the at least one textual content through textual analysis, wherein the method comprises:
interpreting, using the processing device, the at least one textual content through semantic analysis based on the processing; and
detecting, using the processing device, at least one of a fanaticism polarity and a fanaticism intensity of the at least one textual content through sentiment analysis based on the interpreting, wherein the determining of the at least one fanaticism value is based on the detecting.

8. The method of claim 1, wherein the at least one digital content comprises at least one graphical content, wherein the analyzing comprises processing the at least one graphical content through image analysis, wherein the method comprises:
interpreting, using the processing device, the at least one graphical content through image-semantic analysis based on the processing; and
detecting, using the processing device, at least one of a fanaticism polarity and a fanaticism intensity of the at least one graphical content through image-sentiment analysis based on the interpreting, wherein the determining the at least one fanaticism value is based on the detecting.

9. The method of claim 1, wherein the at least one digital content comprises at least one audio content, wherein the analyzing comprises processing the at least one audio content through audio analysis, wherein the method comprises:
interpreting, using the processing device, the at least one audio content through audio-semantic analysis based on the processing; and
detecting, using the processing device, at least one of a fanaticism polarity and a fanaticism intensity of the at least one audio content through audio-sentiment analysis based on the interpreting, wherein the determining the at least one fanaticism value is based on the detecting.

10. The method of claim 1, wherein the at least one digital content comprises at least one video content, wherein the analyzing comprises processing the at least one video content through video analysis, wherein the method comprises:
interpreting, using the processing device, the at least one video content through video-semantic analysis based on the processing; and
detecting, using the processing device, at least one of a fanaticism polarity and a fanaticism intensity of the at least one video content through video-sentiment analysis based on the interpreting, wherein the determining the at least one fanaticism value is based on the detecting.

11. A system for facilitating engagement between entities and individuals, the system comprising:
a communication device configured for:
receiving at least one digital content associated with at least one entity form at least one individual device associated with at least one individual; and
transmitting at least one reward notification to at least one presentation device; and
a processing device configured for:
analyzing the at least one digital content associated with the at least one entity;
determining at least one fanaticism value associated with the at least one digital content based on the analyzing;
determining at least one reward corresponding to the at least one individual based on the at least one fanaticism value; and
generating the at least one reward notification corresponding to the at least one reward based on the determining of the at least one reward.

12. The system of claim 11, wherein the communication device is further configured for receiving at least one sensor data associated with the at least one digital content from at least one sensor, wherein the at least one sensor is configured for generating the at least one sensor data based on at least one of a physical state and a physiological state of the at least one individual, wherein the processing device is further configured for analyzing the at least one sensor data associated, wherein the determining of the at least one fanaticism value is based on the analyzing of the at least one sensor data.

13. The system of claim 12, wherein the at least one sensor is comprised in the at least one individual device.

14. The system of claim 12, wherein the at least one sensor is comprised in at least one wearable device, wherein the at least one individual wears the at least one wearable device.

15. The system of claim 11, wherein the processing device is further configured for comparing the at least one fanaticism value and at least one predetermined fanaticism value, wherein the determining of the at least one reward corresponding to the at least one individual is based on the comparing.

16. The system of claim 11, wherein the processing device is further configured for:
identifying at least one content metadata associated with the at least one digital content; and
analyzing the at least one content metadata, wherein the determining of the at least one fanaticism value is based on the analyzing of the at least one content metadata.

17. The system of claim 11, wherein the at least one digital content comprises at least one textual content, wherein the analyzing comprises processing the at least one textual content through textual analysis, wherein the processing device is further configured for:
interpreting the at least one textual content through semantic analysis based on the processing; and
detecting at least one of a fanaticism polarity and a fanaticism intensity of the at least one textual content through sentiment analysis based on the interpreting, wherein the determining of the at least one fanaticism value is based on the detecting.

18. The system of claim 11, wherein the at least one digital content comprises at least one graphical content, wherein the analyzing comprises processing the at least one graphical content through image analysis, wherein the processing device is further configured for:
interpreting the at least one graphical content through image-semantic analysis based on the processing; and
detecting at least one of a fanaticism polarity and a fanaticism intensity of the at least one graphical content through image-sentiment analysis based on the interpreting, wherein the determining the at least one fanaticism value is based on the detecting.

19. The system of claim 11, wherein the at least one digital content comprises at least one audio content, wherein the analyzing comprises processing the at least one audio content through audio analysis, wherein the processing device is further configured for:
interpreting the at least one audio content through audio-semantic analysis based on the processing; and
detecting at least one of a fanaticism polarity and a fanaticism intensity of the at least one audio content through audio-sentiment analysis based on the interpreting, wherein the determining the at least one fanaticism value is based on the detecting.

20. The system of claim 11, wherein the at least one digital content comprises at least one video content, wherein the analyzing comprises processing the at least one video content through video analysis, wherein the processing device is further configured for:
interpreting the at least one video content through video-semantic analysis based on the processing; and
detecting at least one of a fanaticism polarity and a fanaticism intensity of the at least one video content through video-sentiment analysis based on the interpreting, wherein the determining the at least one fanaticism value is based on the detecting.
